(19) United States
(12) Patent Application Publication
(10) Pub. No.: US 2021/0284952 A1
(43) Pub. Date: Sep. 16, 2021

(54) METHODS OF APPLYING ACETATE TOXICITY AND INDUCING ACETATE UPTAKE IN MICROALGAE CULTURES

(71) Applicant: HELIAE DEVELOPMENT LLC, Gilbert, AZ (US)

(72) Inventors: Eneko GANUZA TABERNA, Tempe, AZ (US); Magdalena Amezquita Ernult, Chandler, AZ (US); Luke Cizek, Mesa, AZ (US); Charles Sellers, Mesa, AZ (US); Kara Paolilli Bautista, Menlo Park, CA (US); Jon Hansen, Tempe, AZ (US)

(21) Appl. No.: 16/336,632

(22) PCT Filed: Sep. 26, 2017
(86) PCT No.: PCT/US2017/053436

§ 371 Date: Mar. 26, 2019

Related U.S. Application Data
(60) Provisional application No. 62/402,794, filed on Sep. 30, 2016.

(51) Int. Cl. C12N 1/12

(52) U.S. Cl. CPC C12N 1/12; C12N 2500/30; C12N 2500/05; C12N 2500/34; C12N 2500/36; C12N 2500/74; C12N 2500/76; C12N 2500/32; C12N 2501/999

(57) ABSTRACT

Methods of culturing microalgae in acetate toxicity conditions to induce the uptake of acetate, control contamination, increase the metabolic rate, increase the respiration rate, increase the accumulation of lipids, and decreasing the accumulation of protein are disclosed. Embodiments include methods of controlling the internal microalgae cell acetate concentration by manipulating the culture pH and residual acetate concentration.

### CROSS REFERENCE TO RELATED APPLICATIONS

This application claims the benefit of U.S. Provisional Patent Application No. 62/402,794, entitled METHODS OF APPLYING ACETATE TOXICITY AND INDUCING ACETATE UPTAKE IN MICROALGAE CULTURES, filed Sep. 30, 2016, the contents of which is hereby entirely incorporated by reference.

### BACKGROUND

The use of acetate and acetic acid as an organic carbon source for microalgae enables the culture to experience the high productivities associated with mixotrophic and heterotrophic cultures. Use of acetate and acetic acid is also known to inhibit contaminating bacteria in a microalgae culture. However, over time the residual acetate concentration of the microalgae can rise to levels that are toxic to the microalgae without careful control. Separate from preventing conditions toxic to microalgae when engaging in efforts to suppress contamination, industrial cultivation of microalgae also requires optimization of the conditions for growth and accumulation of target metabolites for efficient commercial production. A thorough understanding of the microalgae cells metabolism and the interaction between organic carbon uptake, toxicity, cell growth, and metabolite accumulation, may dictate which methods, conditions, and inputs to use for commercial production.

### SUMMARY

Methods of culturing microalgae in acetate toxicity conditions to produce benefit for the microalgae culture may comprise inducing the uptake of acetate, controlling contamination, increasing the metabolic rate, increasing the respiration rate, increasing the accumulation of lipids, and decreasing the accumulation of protein are disclosed. Embodiments include methods of controlling the internal microalgae cell acetate concentration by manipulating the culture pH and residual acetate concentration. The method may be conducted in nitrogen sufficient or nitrogen deficient conditions. The methods may also be used to increase the life of a culture in the presence of refined or unrefined by-product streams from industrial, municipal, or agricultural sources.

### BRIEF DESCRIPTION OF THE FIGURES

FIG. 1 shows a diagram of acetate uptake by a cell.

FIG. 2 shows a graph of the effects of pH control on cellular acetate concentration for Chlorella.

FIG. 3 shows a graph of the dissolved oxygen and pH fluctuations for a microalgae culture.

FIG. 4 shows a graph of pH, dissolved oxygen, residual acetate, and calculated internal acetate for a microalgae culture.

FIG. 5 shows a graph of pH, dissolved oxygen, residual acetate, and calculated internal acetate for a microalgae culture.

FIG. 6 shows graph of internal cell acetate concentration, dry weight and residual acetate in a microalgae culture.

### DETAILED DESCRIPTION

While culturing microalgae in mixotrophic or heterotrophic culture conditions utilizing acetate or acetic acid as an organic carbon source is known, the inventors have developed methods to leverage the acetate toxicity level in a culture of microalgae to induce the uptake of organic carbon for controlling the metabolism of the microalgae while suppressing bacterial contamination. Contemplated benefits of these methods include, but are not limited to: lowering the amount of or inhibiting bacteria in a non-axenic mixotrophic or heterotrophic culture of microalgae; increasing the metabolic rate of the microalgae; increasing the respiration rate of the microalgae; reducing the culturing time for production of specific metabolite (e.g., fatty acid) by the microalgae; increasing the initial rate of growth in a microalgae culture; increasing the accumulation of lipids in nitrogen sufficient conditions: increasing the accumulation of lipids in nitrogen deficient conditions; increasing the maximum culture density in a closed bioreactor culture; improved control over the dissolved oxygen level in a microalgae culture; decreasing the accumulation of protein; decreasing the accumulation of carbohydrates; enabling a microalgae culture to survive in a culture medium comprising refined or unrefined by-product streams from industrial, municipal, or agricultural sources; and enabling a culture of microalgae to extend the culture life in the presence of contaminating organisms or in the event where the culture sterility or axenic conditions are lost. The term “microalgae” refers to any microorganisms classified as microalgae, cyanobacteria, diatoms, dinoflagellates, or other similar single cell microorganisms, whether freshwater or marine, capable of growth in phototrophic, mixotrophic, or heterotrophic culture conditions. In one aspect the microalgae is eukaryotic.

The term “pH auxostat” refers to the microbial cultivation technique that couples the addition of fresh medium (e.g., medium containing organic carbon or acetic acid) to pH control. As the pH drifts from a given set point, fresh medium is added to bring the pH back to the set point. The rate of pH change is often an excellent indication of growth and meets the requirements as a growth-dependent parameter. The feed may keep a residual nutrient concentration (e.g., acetic acid) in balance with the buffering capacity of the medium. The pH set point may be changed depending on the microorganisms present in the culture at the time. The microorganisms present may be driven by the location and season where the bioreactor is operated and how close the cultures are positioned to other contamination sources (e.g., other farms, agriculture, ocean, lake, river, waste water). The rate of medium addition is determined by the substrate consumption rate of the microorganism and the buffering capacity of the media. The pH drift of the culture is mostly driven by the acetic acid consumption and therefore pH auxostat is designed to replace the acetic acid that was consumed and maintaining a constant residual acetate concentration. Because there are other processes other than the acetic acid consumption that affect the medium pH the residual acetate concentration may deviate from the initial set point.

In some embodiments, the inventive method utilizes a pH auxostat to provide multiple functions comprising at least one selected from the group consisting of: supplying acetic acid to the microalgae culture as a source of organic carbon, maintaining the culture pH in a desired range, and maintaining the residual acetate concentration of the culture medium (i.e., acetate toxicity conditions) in a desired range. The toxicity of the environment is governed by a variety of factors, such as but not limited to, the total concentration of acetate in the culture and the pH of the culture; and thus the residual acetate concentration of the culture medium forming the toxicity is controlled by the initial concentration of acetate and the supply of acetic acid through the pH auxostat. Maintaining a residual acetate concentration in the culture medium is not inherent in a pH auxostat system, but the ability to control acetic acid toxicity in a pH auxostat system as developed by the inventors using the described inventive methods may produce the benefits described.

While some microalgae are known to use acetate or acetic acid as a carbon source, the inventors determined that an acetate concentration that is too high can also be toxic to microalgae, and thus acetate tolerance limits may vary among microalgae. The higher tolerance limit of microalgae to acetate and undissociated acetic acid, as compared to bacteria, may be attributable to microalgae having differentiated organelles and a nucleus. However, the indiscriminate use of acetic acid in a microalgae culture contaminated with bacteria may negatively affect the bacteria, but may also be negatively affect the microalgae if the residual acetate concentration formed in the culture medium is above the tolerance limit of the microalgae. Therefore, in some embodiments the developed methods operate inside a defined toxicity window that approaches the acetate tolerance limit of the microalgae in order to control the population so contaminating organisms (e.g., bacteria), and may be achieved by deviating from the convention operation of a pH auxostat system.

The inventors found that excretion of different substances such as organic acids, during different processes, such as ammonia consumption, may decrease the residual acetate concentration of the culture medium while the other processes, such as consumption of nitrates, amino acid production, or protein accumulation, may increase the residual acetate concentration. While not being limited to any particular theory, the inventors found that controlling the factors that affect residual acetate concentration of the culture medium in a pH auxostat system are influential in controlling acetate toxicity, especially when the aim is to operate in the defined acetate toxicity window that favors the growth of microalgae over contaminating organisms (e.g., bacteria). Therefore, realizing the benefits of acetate toxicity in a microalgae culture may comprise developing the techniques for a tight acetate and pH control.

In some embodiments, the inventive method utilizes a pH auxostat to provide a supply of at least one of acetate and acetic acid to the microalgae culture as a source of organic carbon, a method of maintaining the culture pH in a desired range, and a method of maintaining the residual acetate concentration of the culture medium (i.e., acetate toxicity conditions) in a desired range. In some embodiments, the pH auxostat system may comprise a solenoid valve, a peristaltic pump, a pH probe and a pH controller. In some embodiments, the pH auxostat system may comprise a drip application device controlled by a needle valve, a metering pump or a peristaltic pump, and a pH controller. The pH controller may be set at a threshold level (i.e., set point) and activate the auxostat system to supply acetic acid to the culture when the measured pH level is above the set threshold level. The frequency of pH measurements, administration of acetic acid by the auxostat system, and mixing of the culture are controlled in combination to keep the pH value substantially constant. In some embodiments, the acetic acid feed may be diluted in water to a concentration below 100% and as low as 0.5%, with a preferable concentration between 15% and 50%. In other embodiments the acetic acid may be at concentrations below 10% in order to continuously dilute the culture of microalgae. In other embodiments, the acetic acid may be mixed together with other nutrient media, acids, or organic carbon sources.

Non-limiting examples of suitable microalgae for mixotrophic or heterotrophic growth using acetic acid or acetate as an organic carbon source may comprise microalgae of the genera: Chlorella, Anacystis, Synechococcus, Synechocystis, Neospongiococcum, Chlorococcum, Phaeodactylum, Spirulna, Micractinium, Haematococcus, Nannochloropsis, Brachiomonas, Schizochvtrium, Aurantiochytrium, Crypthecodinium, Chlamyidomonas, Euglena, and species thereof. Non-limiting examples of other mixotrophic or heterotrophic capable microalgae may comprise: Tetraselmis, Nitzschia, Galdieria, Agmenellum, Goniotrichium. Navicula. Phaeodactylum, Rhodomonas, Cyclotella, Skeletonema, Pavlova, Dunaliella, and species thereof. A culture of microalgae can comprise combinations of two or more of any of these listed types of organisms and/or the other types of organisms described in connection with the term “microalgae” above. The culture can also or alternatively be characterized by lacking the inclusion of one or more of any such types of organisms.

Other organic carbon sources suitable for growing microalgae mixotrophically or heterotrophically may comprise: ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, xylose, and combinations thereof. The organic carbon source may comprise any single source, combination of sources, and dilutions of single sources or combinations of sources.

Analysis of the DNA sequence of the strain of Chlorella sp. HS26 described in the specification was done in the NCBI 18s rDNA reference database at the Culture Collection of Algae at the University of Cologne (CCAC) showed substantial similarity (i.e., greater than 95%) with multiple known strains of Chlorella and Micractimaum. Those of skill in the art will recognize that Chlorella and Micractinium appear closely related in many taxonomic classification trees for microalgae, and strains and species may be re-classified from time to time. Thus for references throughout the instant specification for Chlorella sp. HS26, it is recognized that microalgae strains in related taxonomic classifications with similar characteristics to the reference Chlorella strain would reasonably be expected to produce similar results.

Additionally, taxonomic classification has also been in flux for microalgae in the genus Schizochytrium. Some organisms previously classified as Schizochytrium have been reclassified as Aurantiochytrium, Thraustochytrium, or Oblongichytrium. See Yokoyama et al. Taxonomic rearrangement of the genus Schizochytrium sensu lato based on morphology, chemotaxonomic characteristics, and 18S rRNA gene phylogeny (Thrausochytriaceae, Labyrinthulomycetes): emendation for Schizochytrium and erection of Aurantiochytrium and Oblongichytrium gen. nov. Mycoscience (2007) 48:199-211. Those of skill in the art will recognize that Schizochytrium, Aurannochytrium, Thraustochytrium, and Oblongichytrium appear closely related in many taxonomic classification trees for microalgae, and strains and species may be re-classified from time to time. Thus for references throughout the instant specification for Schizochytrium, it is recognized that microalgae strains in related taxonomic classifications with similar characteristics to Schizochytrium would reasonably be expected to produce similar results.

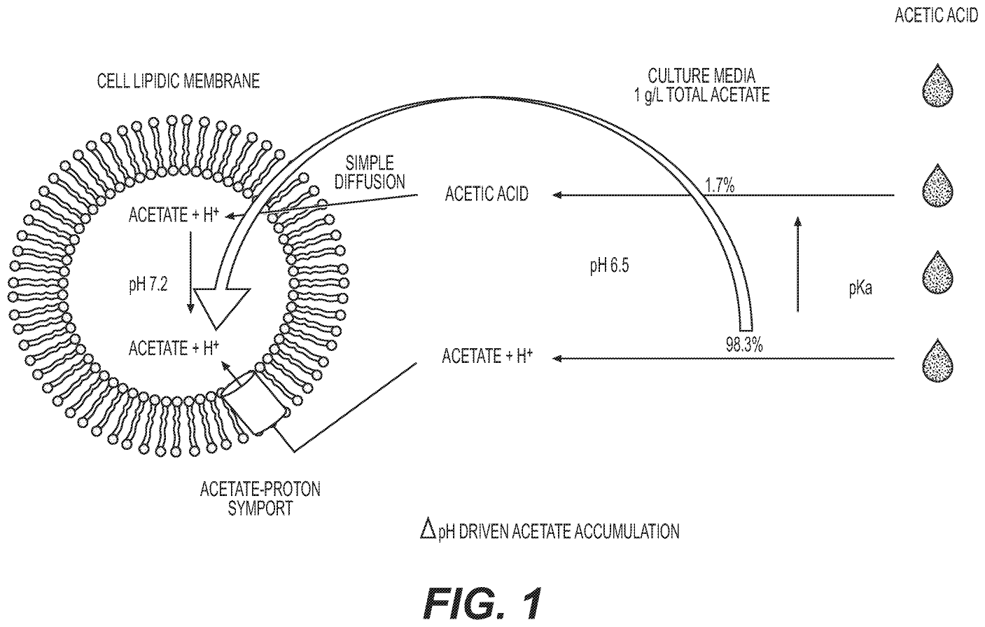

Without being bound by any particular theory, the inventors postulate that the accumulation of acetate inside a cell is driven by the pH gradient between the internal cell pH and pH of the culture medium outside the cell. In further explanation, acetate and free protons enter microalgae cells through an active symport transporter, while acetic acid is membrane permeable and may diffuse passively into the microalgae cell. Together these characteristics allow the he uptake of acetate to be controlled by the cell, but not the diffusion of acetic acid. As shown in FIG. 1, when the culture medium pH is below neutral (i.e., outside of about 7-8), the intra and extracellular dissociation equilibrium along with diffusion equilibrium of acetic acid across the cell membrane results in an intracellular concentration of acetate greater than the residual acetate concentration in the culture medium. As further described in FIG. 1, the concentration of acetate in a culture medium will convert to acetic acid as the culture medium pH decreases and approaches the pKa value of acetic acid (about 4.7). The increase of available acetic acid in the culture medium may increase the diffusion of acetic acid through the cell membrane and into the cell with an internal pH higher than the culture medium pH. Due to the higher pH value within the cell than outside cell, at least some of the acetic acid will convert into acetate and a free proton, which increases the acetate concentration within the cell and decrease the internal cell pH. The inventors hypothesize that the concentration of acetate in the cell may be controlled by manipulating the internal cell and culture medium pH gradient (i.e., intra/extracellular pH gradient, and that the acetate toxicity will be proportional to the internal cell acetate concentration.

Thus acetic acid may become toxic to microalgae when the pH gradient between the internal cell pH and pH of the culture medium outside the cell induces the built up of acetate inside the cells. Because the microalgae pH homeostasis will tend to maintain an internal cell pH slightly above neutral (about 7-8) in response to medium acidification, the acetate built up inside the cell may be modeled. The internal acetate concentration of a cell may be calculated from the external culture pH, and the residual acetate concentration in the culture, assuming that the internal pH of the cell and the ionic strength are maintained constant. The pH gradient between the internal cell pH and pH of the culture medium outside the cell may be calculated with the following equation derived from the Hendersen Hassleback equation, from which the internal acetate concentration can be solved:

$$Δ pH = pHi - pHo = log \frac{( [ A ] i + [ AH ] I )}{( [ A ] O + [ AH ] 0 )}$$

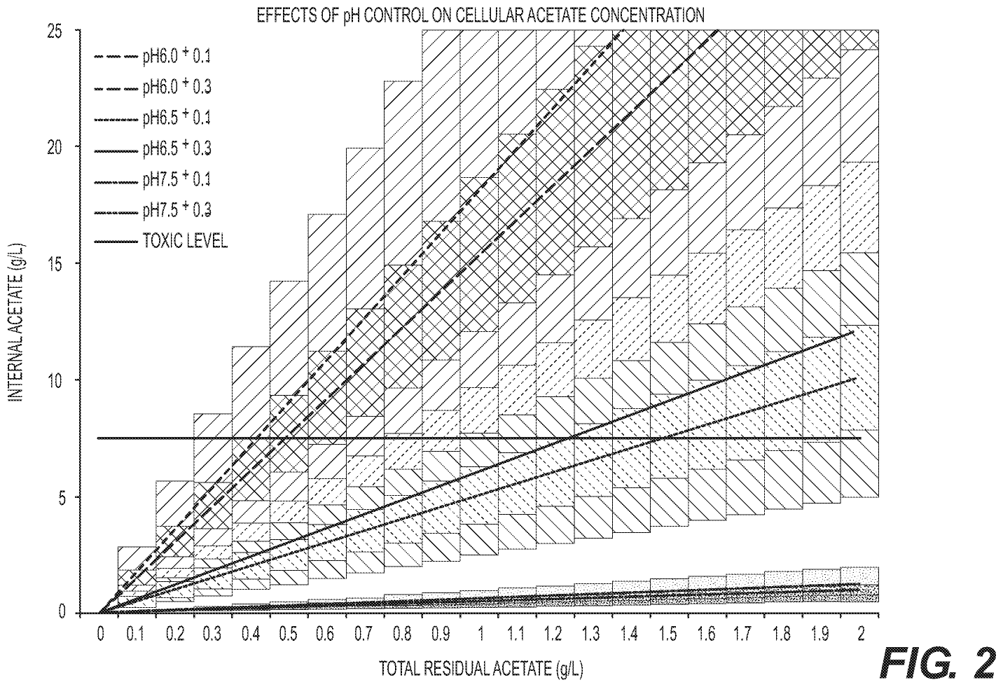

pHi=pH inside the cell, pHo=pH outside the cell, AH=acetic acid, A=acetate, O=outside cell, I=inside cell. The relationship may be illustrated with the non-limiting examples of: at 1 g/L concentration of residual acetate in a microalgae culture at a pH of 8.5 results in a low concentration of acetate within the cell, but a 1 g/L concentration of residual acetate in a culture at a pH of 6.5 results in near toxic concentration of acetate in the cell. For example, the impact of the pH control amplitude at different pH set points for culture medium with different acetate concentrations (i.e., total residual acetate) were modeled for the determined toxicity limit for Chlorella sp. HS26 (7.5 g/L internal acetate) are shown in Table 1 and FIG. 2, including a horizontal line indicating the 7.5 g/L internal acetate toxicity limit in FIG. 2). This modelling methodology may be applied microalgae generally for microalgae where the acetate toxicity limit is determined.

TABLE 1
Predicted values of internal acetate built up as a function
of pH and intracellular acetate concentration.

| Medium | Intracellular Acetate Concentration (g/L) | | |
|---|---|---|---|
| Acetate | | Medium pH | |
| Concentration | 7.0 | 6.5 | 6.0 |
| 0 | 0.0 | 0.0 | 0.0 |
| 1 | 1.6 | 4.9 | 15.0 |
| 2 | 3.2 | 9.9 | 30.0 |
| 3 | 4.7 | 14.8 | 45.1 |
| 4 | 6.3 | 19.7 | 60.1 |
| 5 | 7.9 | 24.6 | 75.1 |
| 6 | 9.5 | 29.6 | 90.1 |
| 7 | 11.0 | 34.5 | 105.2 |
| 8 | 12.6 | 39.4 | 120.2 |
| 9 | 14.2 | 44.3 | 135.2 |
| 10 | 15.8 | 49.3 | 150.2 |
| 11 | 17.3 | 54.2 | 165.3 |
| 12 | 18.9 | 59.1 | 180.3 |
| 13 | 20.5 | 64.0 | 195.3 |
| 14 | 22.1 | 69.0 | 210.3 |
| 15 | 23.6 | 73.9 | 225.4 |

As demonstrated in FIG. 2, the inventors determined controlling the acetate toxicity in a Chlorella sp. HS26 microalgae culture may comprise maintaining a constant pH and maintaining constant residual acetate levels in the culture medium. Control over these parameters may aid in dictating the amount of diffusion of acetic acid occurring through the microalgae cell membrane. Relevant constraints for controlling the pH may comprise, but are not limited to, the scale (e.g., size, depth, volume) of the bioreactor, the location of introduction of acetic acid from a dosing system (e.g., pH auxostat), the pH control PID, amplitude, the peristaltic pump size and duty cycle for the dosing system, the aqueous culture medium buffering capacity, and the acetic acid concentration in the dosing feedstock.

In some embodiments, the residual acetate concentration in the microalgae culture medium may be controlled through the addition of an acid other than acetic acid, such as but not limited to hydrochloric acid (HCl), phosphoric acid (H₃PO₄), and sulfuric acid (H₂SO₄). The additional acid may provide the function of lowering the pH of the culture, decreasing the residual acetate concentration, or avoiding the increase of residual acetate in the system. In some embodiments, the medium formulation may be changed by increasing and decreasing the concentration of the nutrients other than acetic acid or, replacing the type nitrogen source fed to the reactor. In some embodiments, the nitrogen source may comprise at least one of monosodium glutamate, ammonia, ammonium (e.g., ammonium hydroxide, ammonium phosphate, ammonium acetate), nitrates, urea, glycine, and combinations thereof. In some embodiments, the residual acetate concentration may be intentionally increased in order to induce the uptake of acetic acid by the cells through diffusion through the microalgae cell membrane, and thus increase the respiration of the cells. The increased respiration of the cells may correspond to a decrease in the dissolved oxygen concentration of the microalgae culture.

In some embodiments, the acetate toxicity threshold level of microalgae may vary based on the type microalgae and the pH of the culture. In some embodiments, the acetate toxicity threshold level of Chlorella may be in the range of 5,000 to 7,000 ppm at a pH of about 7.2, which is the equivalent range of about 6.9 to 10.4 g/L of acetate. This toxicity threshold of acetate by Chlorella is two magnitudes of order less than the toxicity threshold of glucose for Chlorella. In some embodiments, the acetate toxicity threshold level of Aurantiochytrium may be in the range of about 50 to 150 g/L acetate at a pH of about 7.0. Within this acetate toxicity threshold range (i.e., window) the growth curve and dry weights of the microalgae start to show negative effects from acetate. This large discrepancy in toxicity concentrations between acetate and glucose may be explained by the pH gradient built up of acetate by microalgae cells.

In some embodiments, the acetate toxicity threshold level of Chlorella at a pH of about 7 may comprise an internal acetate concentration in the range of 6 to 11 g/L. In some embodiments, the acetate toxicity threshold level of Chlorella at a pH of about 7 may comprise an internal acetate concentration in the range of 6 to 7 g/L. In some embodiments, the acetate toxicity threshold level of Chlorella at a pH of about 7 may comprise an internal acetate concentration in the range of 7 to 8 g/L. In some embodiments, the acetate toxicity threshold level of Chlorella at a pH of about 7 may comprise an internal acetate concentration in the range of 8 to 9 g/L. In some embodiments, the acetate toxicity threshold level of Chlorella at a pH of about 7 may comprise an internal acetate concentration in the range of 9 to 10 g/L. In some embodiments, the acetate toxicity threshold level of Chlorella at a pH of about 7 may comprise an internal acetate concentration in the range of 10 to 11 g/L.

In some embodiments, the acetate toxicity threshold level of Aurantiochytrium at a pH of about 7 may comprise an internal acetate concentration in the range of 50 to 150 g/L. In some embodiments, the acetate toxicity threshold level of Aurantiochytrium at a pH of about 7 may comprise an internal acetate concentration in the range of 50 to 60 g/L. In some embodiments, the acetate toxicity threshold level of Aurantiochytrium at a pH of about 7 may comprise an internal acetate concentration in the range of 60 to 70 g/L. In some embodiments, the acetate toxicity threshold level of Aurantiochytrium at a pH of about 7 may comprise an internal acetate concentration in the range of 70 to 80 g/L. In some embodiments, the acetate toxicity threshold level of Aurantiochytrium at a pH of about 7 may comprise an internal acetate concentration in the range of 80 to 90 g/L. In some embodiments, the acetate toxicity threshold level of Aurantiochytrium at a pH of about 7 may comprise an internal acetate concentration in the range of 90 to 100 g/L. In some embodiments, the acetate toxicity threshold level of Aurantiochytrium at a pH of about 7 may comprise an internal acetate concentration in the range of 100 to 110 g/L. In some embodiments, the acetate toxicity threshold level of Aurantiochytrium at a pH of about 7 may comprise an internal acetate concentration in the range of 110 to 120 g/L. In some embodiments, the acetate toxicity threshold level of Aurantiochytrium at a pH of about 7 may comprise an internal acetate concentration in the range of 120 to 130 g/L. In some embodiments, the acetate toxicity threshold level of Aurantiochytrium at a pH of about 7 may comprise an internal acetate concentration in the range of 130 to 140 g/L. In some embodiments, the acetate toxicity threshold level of Aurantiochytrium at a pH of about 7 may comprise an internal acetate concentration in the range of 140 to 150 g/L.

In some embodiments, a method of culturing microalgae in medium or with a feedstock comprising a low cost refined or unrefined by-product stream from industrial (e.g., manufacturing; carpet, textile, pulp, or paper milling), municipal (e.g., sewage), or agricultural (e.g., feed lots, field runoff) sources may further comprise a supply of at least one of acetic acid, acetate, and another organic carbon source. In some embodiments, the refined or unrefined by-product stream from industrial, municipal, or agricultural sources may comprise: ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, yeast extract, xylose, woody biomass, lignocellulosic biomass, food waste, beverage waste, pigments, nitrates, phosphates, phosphites, and combinations thereof. In some embodiments, the acetate toxicity of a microalgae culture comprising refined or unrefined by-product stream from industrial, municipal, or agricultural sources may be controlled as described through the instant specification to increase the culture life of the microalgae while suppressing completion from contaminating organisms (e.g., bacteria). In some embodiments, the acetate toxicity of a culture of microalgae comprising refined or unrefined by-product stream from industrial, municipal, or agricultural sources may be controlled in bioreactor systems that are open or closed.

In some embodiments, a method of culturing microalgae with acetate or acetic acid in which at least one of the residual acetate and culture medium pH is controlled to maintain a desired range of acetate toxicity may be used in a microalgae culture in non-axenic conditions (e.g., culture experiencing bacterial contamination). In some embodiments, a method of culturing microalgae with acetate or acetic acid in which at least one of residual acetate and culture medium pH is controlled to maintain a desired range of acetate toxicity may be used in a microalgae culture in axenic conditions to mitigate any detrimental effects that may occur from a system breach or equipment failure in which the axenic conditions of the microalgae culture are compromised.

### EXAMPLES

Embodiments of the invention are exemplified and additional embodiments are disclosed in further detail in the following Examples, which are not in any way intended to limit the scope of any aspect of the invention described herein.

### Example 1

Cultures of Chlorella sp. HS26 were prepared to determine the tolerance to different concentrations of sodium acetate (sodium salt of acetic acid) on mixotrophically cultured Chlorella at a constant culture medium pH. 100 mL volume cultures were prepared in 250 mL flasks, and cultured at a pH of 7.5, temperature of 25° C., a shaking frequency of 100 rpm, and a light intensity of 100 μM photon/m² s for 7 days (168 hours). The cultures were inoculated into two times BG-11 culture media at a cell density of 1.12 g/L. The different treatments of sodium acetate consisted of concentrations of 0, 2.5, 5, 7.5, 10, 20, 30, and 40 g/L. Samples were taken every 24 hours to measure the cell dry weights (g/L) of the cultures. The results of the experiment are presented in Table 2.

TABLE 2

| | Chlorella dry weights (g/L) of treatments | | | | | | | |
|---|---|---|---|---|---|---|---|---|
| Time | by Concentration of Sodium Acetate (g/L) | | | | | | | |
| (h) | 0 | 2.5 | 5 | 7.5 | 10 | 20 | 30 | 40 |
| 0 | 0.12 ± | 0.12 ± | 0.12 ± | 0.12 ± | 0.12 ± | 0.12 ± | 0.12 ± | 0.12 ± |
| | 0.00 | 0.00 | 0.00 | 0.00 | 0.00 | 0.00 | 0.00 | 0.00 |
| 24 | 0.20 ± | 0.23 ± | 0.19 ± | 0.17 ± | 0.13 ± | 0.16 ± | 0.14 ± | 0.11 ± |
| | 0.02 | 0.01 | 0.06 | 0.04 | 0.00 | 0.02 | 0.03 | 0.01 |
| 48 | 0.58 ± | 0.85 ± | 0.25 ± | 0.15 ± | 0.16 ± | 0.13 ± | 0.11 ± | 0.09 ± |
| | 0.04 | 0.01 | 0.03 | 0.02 | 0.01 | 0.01 | 0.02 | 0.01 |
| 72 | 0.77 ± | 1.10 ± | 0.55 ± | 0.13 ± | 0.11 ± | 0.12 ± | 0.09 ± | 0.10 ± |
| | 0.02 | 0.05 | 0.06 | 0.03 | 0.03 | 0.02 | 0.01 | 0.04 |
| 144 | 2.56 ± | 2.72 ± | 1.89 ± | 0.10 ± | 0.09 ± | 0.07 ± | 0.08 ± | 0.09 ± |
| | 0.23 | 0.06 | 0.19 | 0.09 | 0.03 | 0.00 | 0.01 | 0.00 |
| 168 | 3.09 ± | 3.23 ± | 2.41 ± | 0.07 ± | 0.00 ± | 0.00 ± | 0.00 ± | 0.00 ± |
| | 0.03 | 0.04 | 0.09 | 0.00 | 0.00 | 0.00 | 0.00 | 0.00 |

The results in Table 2 show that the Chorella grew well on 2.5 g/L sodium acetate (0.620 g/L day) and showed positive growth on 5 g/L (0.065 g/L day), but did not show any growth on concentrations of 7.5 g/L and higher. The culture that received 2.5 g/L of sodium acetate also reached exponential phase after 48 hours. Thus, the acetate concentration tolerance for Chlorella was determined to be approximately 7.5 g/L of sodium acetate in the culture for the condition of a pH of 7.5, which may be used to determine the boundary of the acetate toxicity range for inducing uptake of acetate in the cells.

### Example 2

This experiment was conducted to determine the effect of different culture medium pH levels on the growth of Chlorella sp. HS26 at a constant culture medium acetate concentration. Duplicate 100 ml flasks of axenic cultures of Chlorella were adjusted to initial pH levels of 2.5, 3.5, 4.5, 5.5, 6.5, 7.0, 7.5, 8.5, 9.5, and 10.5. The culture pH was adjusted using either hydrochloric acid (HCl) or sodium hydroxide (NaOH). All flask cultures were fed 2.4 g/L concentration sodium acetate (equivalent to about 1 g/L acetate). Samples of the flask culture were taken initially and every other day over a six day period (144 hours). The results of the cell dry weight (g/L) analysis are presented in Table 3, with n.d. denoting where a value was too low to be detected. It was noted during the experiment that the cultures with the higher initial pH values (i.e., 6.5 and above) equilibrate to about 7.5 within 24 hours, and to about 8.2 within 48 hours, while the lower initial pH values (i.e., below 6.5) were able to maintain the initial pH value for at least 24 hours. All flasks were able to maintain a pH value within a tolerance of 0.15 (+/−) for at least 3 hours.

TABLE 3

| Time | Cell Dry Weight (g/L) of Chlorella by initial culture pH | | | | | | | | |
|---|---|---|---|---|---|---|---|---|---|
| (h) | 2.5 | 3.5 | 4.5 | 5.5 | 6.5 | 7.5 | 8.5 | 9.5 | 10.5 |
| 0 | 0.6 ± | 0.6 ± | 0.6 ± | 0.6 ± | 0.6 ± | 0.6 ± | 0.6 ± | 0.6 ± | 0.6 ± |
| | 0.0 | 0.1 | 0.0 | 0.0 | 0.0 | 0.0 | 0.0 | 0.0 | 0.0 |
| 24 | 0.5 ± | 0.5 ± | 0.5 ± | 0.6 ± | 0.8 ± | 0.6 ± | 0.8 ± | 0.8 ± | 0.9 ± |
| | 0.0 | 0.0 | 0.0 | 0.0 | 0.1 | 0.3 | 0.0 | 0.0 | 0.0 |
| 48 | 0.5 ± | 0.5 ± | 0.4 ± | 0.6 ± | 1.8 ± | 1.7 ± | 2.4 ± | 1.2 ± | 1.4 ± |
| | 0.0 | 0.1 | 0.1 | 0.0 | 0.2 | 0.2 | 0.2 | 0.1 | 0.1 |
| 96 | n.d. | n.d. | n.d. | 0.4 ± | 4.3 ± | 4.4 ± | 4.2 ± | 4.3 ± | 4.3 ± |
| | | | | 0.0 | 0.2 | 0.0 | 0.0 | 0.2 | 0.1 |
| 144 | n.d. | n.d. | n.d. | n.d. | 6.0 ± | 6.0 ± | 6.0 ± | 6.1 ± | 6.1 ± |
| | | | | | 0.1 | 0.0 | 0.4 | 0.1 | 0.1 |

The results in Table 3 show that the cell dry weight did not increase over time for cultures at a pH of 5.5 or lower, indicating that productive Chlorella cultures should be cultured at a pH above 5.5 when culturing with the given sodium acetate concentration. The cultures at pH of 6.5 and above showed productive growth beginning at 48 hours after inoculation. The results also showed that the Chlorella was able to survive to some degree at pH values of 5.5 and lower but experienced negative effects from acetate toxicity. Under the tested conditions the calculated internal cell acetate concentration (5 g/L) matches with the tolerance limit calculated in Example 1. Therefore, as predicted in the previously described model the proposed the acetate toxicity can be controlled by either by increasing the acetate concentration in the culture medium or decreasing the culture medium pH. From the results of Examples 1 and 2, the acetic acid toxicity may be more efficiently induced by pH manipulation in a commercial process rather than manipulating the acetate concentration.

### Example 3

Cultures of Aurantiochytrium sp. HS399 were prepared to determine the tolerance to different concentrations of sodium acetate (sodium salt of acetic acid) on heterotrophically cultured Aurantiochytrium. 100 mL volume cultures were prepared in 250 mL flasks, and cultured at a pH of 7.0, temperature of 27° C., a shaking frequency of 180 rpm, in the dark for 5 days (120 hours). The cultures were inoculated at the same cell density. The different treatments of sodium acetate consisted of concentrations of 0, 2.5, 5, 10, 20, 40 and 100 g/L. Samples were taken every 24 hours to measure the cell dry weights (g/L) of the cultures. The results of the experiment are presented in Table 4.

TABLE 4

| | Cell Dry Weight (g/L) of Aurantiochytrium | | | | | |
|---|---|---|---|---|---|---|
| Time | by Concentration (g/L) of Sodium Acetate | | | | | |
| (h) | 0 | 5 | 10 | 20 | 40 | 100 |
| 0 | 2.7 ± | 2.7 ± | 2.7 ± | 2.7 ± | 2.7 ± | 2.7 ± |
| | 0.0 | 0.0 | 0.0 | 0.0 | 0.0 | 0.0 |
| 24 | 8.51 ± | 8.01 ± | 8.29 ± | 7.35 ± | 7.06 ± | 1.7 ± |
| | 0.23 | 0.3 | 0.19 | 0.07 | 0.8 | 0.0 |
| 40 | 8.55 ± | 8.13 ± | 9.69 ± | 10.45 ± | 11.84 ± | 0.7 ± |
| | 0.04 | 0.53 | 0.27 | 0.32 | 0.34 | 0.0 |
| 72 | 7.79 ± | 7.49 ± | 10.35 ± | 13.7 ± | 15.78 ± | — |
| | 0.05 | 0.55 | 0.67 | 1.7 | 0.74 | |
| 96 | 7.73 ± | 7.38 ± | 10 ± | 13.11 ± | 18.9 ± | — |
| | 0.21 | 0.46 | 0.21 | 0.19 | 0.95 | |
| 120 | 7.49 ± | 7.6 ± | 10.9 ± | 13.55 ± | 19.65 ± | — |
| | 0.19 | 0.46 | 0.57 | 1.2 | 2.4 | |

The results in Table 4 show that the Aurantiochytrium grew well on concentrations of sodium acetate below 100 g/L. Thus, the acetate concentration tolerance for Aurantiochytrium was determined to be around 100 g/L of sodium acetate in the culture for the condition of a pH of 7.0, which may be used to determine the boundary of the acetate toxicity range for inducing uptake of acetate in the cells.

### Example 4

This experiment was conducted to determine the effect of different culture pH levels on the growth of Aurantiochytrium sp. HS399. Duplicate 100 ml flasks of axenic cultures of Aurantiochytrium were adjusted to initial pH levels of 4, 5, 6, 7, and 8. The culture pH was adjusted using either hydrochloric acid (HCl) or potassium hydroxide (KOH). All flask cultures were fed 2 or 0 g/L concentration sodium acetate (equivalent to 1 g/L acetate). Samples of the flask culture were taken initially and every other day over a five-day period (120 hours). The results of the cell dry weight (g/L) analysis are presented in Table 5. All flasks were able to maintain a pH value within a tolerance of 0.15 (+/−) for at least 3 hours.

TABLE 5

| | Cell Dry Weight (g/L) of Aurantiochytrium by pH | | | | | | |
|---|---|---|---|---|---|---|---|
| Time | 2 g/L Na Acetate | | | | | 0 g/L Na Acetate | |
| (h) | 4 | 5 | 6 | 7 | 8 | 4 | 7 |
| 0 | 0.28 ± | 0.25 ± | 0.18 ± | 0.3 ± | 0.25 ± | 0.28 ± | 0.43 ± |
| | 0.04 | 0.14 | 0.11 | 0.14 | 0.0 | 0.04 | 0.18 |
| 24 | 1.1 ± | 5.15 + | 5.1 ± | 12.25 ± | 9.1 ± | 4.15 ± | 6.7 ± |
| | 0.85 | 1.34 | 3.0 | 0.35 | 2.83 | 0.07 | 1.41 |
| 48 | 0.79 ± | 15.2 ± | 11.55 ± | 10.8 ± | 8.95 ± | 8.85 ± | 15.9 ± |
| | 0.73 | 1.41 | 0.78 | 0.0 | 0.07 | 0.35 | 1.7 |
| 72 | 0.51 ± | 19.4 ± | 16 ± | 15.7 ± | 12.6 ± | 15.1 ± | 16.45 ± |
| | 0.04 | 0.57 | 0.71 | 0.0 | 0.14 | 3.68 | 0.35 |

The results in Table 5 show that the cell dry weight did not increase over time for cultures at a pH of 4, indicating that productive Aurantiochytrium cultures should be cultured at a pH above 4 when culturing with the given sodium acetate concentration. The results also showed that the Aurantiochytrium was able to survive to some degree at pH of 4 but experienced negative effects from acetate toxicity. Similar to Examples 1 and 2, the results of Examples 3 and 4 demonstrate the acetate toxicity can be controlled by either by increasing the acetate concentration in the culture medium or decreasing the culture medium pH.

### Example 5

An experiment was performed to determine the effect on respiration in microalgae Chlorella sp. HS26 in response to acetate toxicity conditions (i.e., the increase of the internal cell acetate concentration). The test was carried out in a culture of Chlorella that was grown mixotrophically utilizing an acetic acid pH auxostat. Data presented corresponds to a continuous 200 L bubble column culture growing in axenic conditions at culture density of 12 g/L, that was incubated at a temperature of 25 f 0.3° C., and aerated at a rate of 30 LPM. The pH probe: E&H, Digital non-glass pH sensor, Tophit CPS471D helped to maintain the pH at 6±0.2 in a pH auxostat mode. The DO was recorder with a Hamilton, EasyFerm Plus Arc 120, P/N: 242091/06 probe. Samples were taken before and after each pulse and analyzed for residual acetate using HPLC. Internal acetate was fluctuations were calculated based on medium residual acetate by integrating the previously presented equation:

$$Δ pH = pHi - pHo = log \frac{( [ A ] i + [ AH ] I )}{( [ A ] O + [ AH ] 0 )}$$

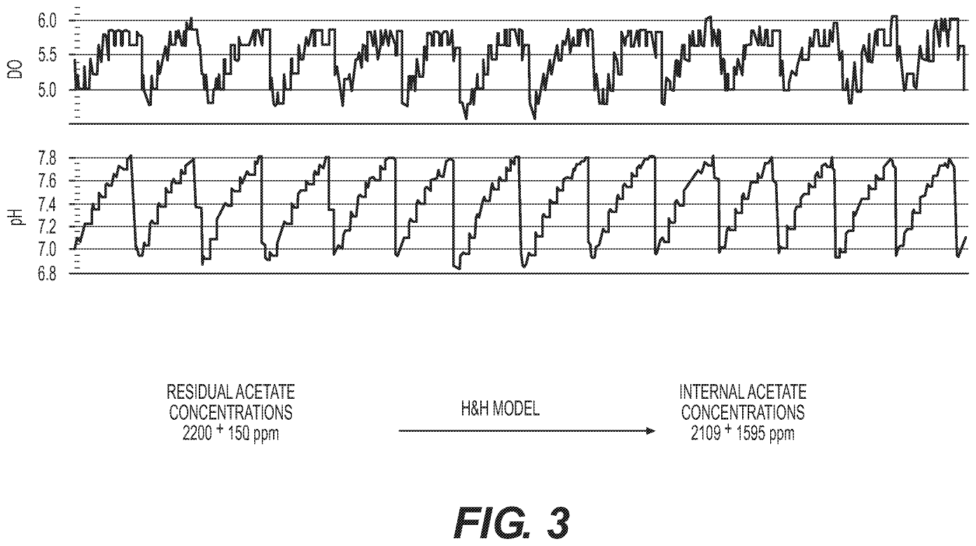

The theoretical internal acetate concentration was calculated by assuming that intracellular internal pH of the cells was regulated to 7.2, that the ionic strength of the media and acetic acid pKa are 0.033 and 4.74 respectively. FIG. 3 shows that dissolved oxygen (DO) fluctuates in response to pH, and appears to correlate to the internal acetate concentration. Based on those results it was concluded that Chlorella increases its respiration rate in response to acetic acid toxicity and therefore acetic acid uptake by the cells can be induced to regulate the cell metabolism.

### Example 6

An experiment was performed to determine the effect on respiration in microalgae Aurantiochytrium sp. HS399 in response to acetate toxicity (i.e., the increase of the internal cell acetate concentration). The test was carried out in a culture of Aurantiochytrium that was grown utilizing an acetic acid pH auxostat. Data presented corresponds to a 4 day old 30 L bubble column bag bioreactor growing in axenic conditions at culture density of 24 g/L, that was incubated at a temperature of 29.5±0.3° C., and aerated at a rate of 10 LPM. The pH probe: E&H, Digital non-glass pH sensor, Tophit CPS471D helped to maintain the pH at 6±0.2 in a pH auxostat mode. The DO was recorder with a Hamilton, EasyFerm Plus Arc 120, P/N: 242091/06 probe. Samples were taken before and after each pulse and analyzed for residual acetate using HPLC. Internal acetate was fluctuations were calculated based on medium residual acetate by integrating the previously presented equation:

$$Δ pH = pHi - pHo = log \frac{( [ A ] i + [ AH ] I )}{( [ A ] O + [ AH ] 0 )}$$

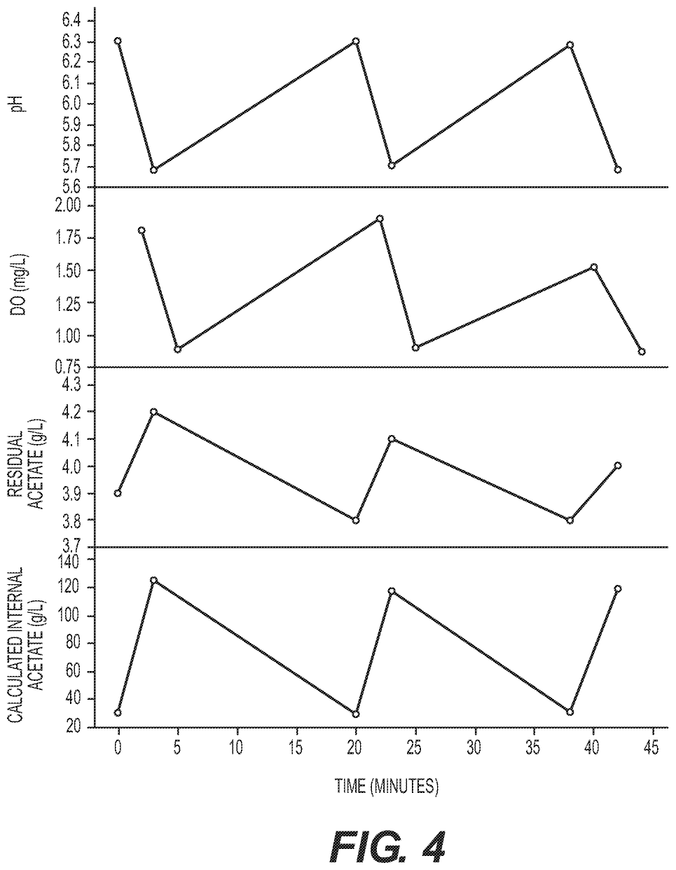

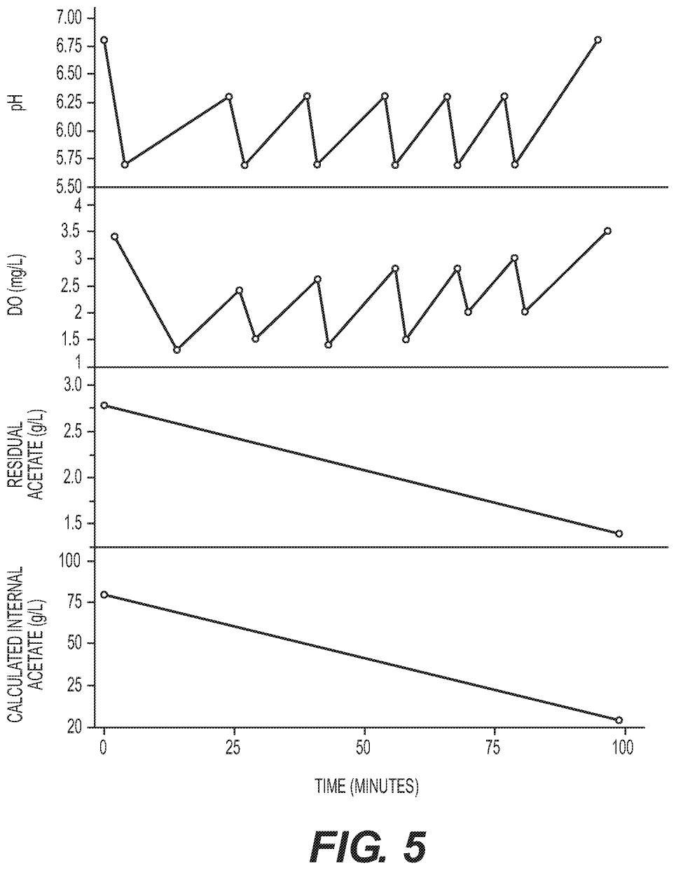

The theoretical internal acetate concentration was calculated by assuming that intracellular internal pH of the cells was regulated to 7.2, that the ionic strength of the media and acetic acid pKa are 0.033 and 4.74 respectively. FIG. 4 shows that dissolved oxygen (DO) fluctuates in response to pH, and appears to correlate to the internal acetate concentration, as the residual culture medium acetate concentration fluctuations were below 2% of its value. Such assumption was confirmed by inducing the same pH-DO fluctuation pattern using HCl/pH control for a few pulses instead of acetic acid as shown in FIG. 5. Based on those results it was concluded that Auraniochytrium increases respiration rate in response to acetic acid toxicity and therefore acetic acid uptake by the cells can be induced to regulate the cell metabolism.

### Example 7

An experiment was conducted to determine the acetate toxicity of Aurantiochytrium sp. HS399. Aurantiochytrium was grown in an acetic acid/pH-auxostat mode in 700 ml bubble column at 27° C. in sterile conditions. It should be noted that those of ordinary skill will recognize that the term “sterile” can be used when the precise intended meaning is “axenic” (meaning that only target microorganism(s) are present in detectable amounts in the culture). In other cases, context will make it clear that the term “sterile” may mean devoid of detectable amounts of living/viable microorganisms. Here, the term is used in the former (axenic) sense. The initial culture medium was supplemented and 0.5 g/L sodium acetate and no other organic carbon source. The base medium contained (g/L): sodium acetate (1), monosodium glutamate monohydrate (2.5) NaCl (12.5). MgSO4-7H2O (2.5), KCl (0.5). CaCl₂) (0.1), KH2PO4 (0.125) vitamins (0.25 ml/L) and trace metal (1.25 ml/L). Trace and vitamins stocks were prepared according to Ashford, et al. 2000. Lipids 35, 1377-1386 The residual acetic acid was maintained at 1±0.5 g/L and the impact of acetate toxicity on the microalgae growth was studied at a pH set point of 5, 4.5 or 7. Cell dry weights and residual acetate concentration in the culture medium were measured daily, and average internal cell acetate concentration was calculated using the previously described model, and the results are shown in Tables 6, 7, and 8.

TABLE 6
Theoretical Average internal acetate. Based on
an assumed constant intracellular pH and the
residual acetate concentration and medium pH.

Averaged over days 1-6

| | Average residual | Average internal |
|---|---|---|
| Treatment | Acetate (g/L) | Acetate (g/L) |
| pH 7.0 | 1.0 | 1.576 |
| pH 5.5 | 1.25 | 53.37 |
| pH 5.0 | 1.5 | 153.42 |
| pH 4.5 | 2.0 | 366.12 |

TABLE 7
Cell Dry Weights (g/L)

pH

| Time (d) | 4.5 | 5.0 | 5.5 | 7.0 |
|---|---|---|---|---|
| 0 | 0.4 | 0.4 | 0.4 | 0.4 |
| 1 | 0.5 ± | 2.3 ± | 2.7 ± | 1.5 ± |
| | 0.0 | 0.1 | 0.0 | 2.1 |
| 2 | 0.3 ± | 5.0 ± | 5.6 ± | 2.9 ± |
| | 0.1 | 0.2 | 0.1 | 4.1 |
| 3 | 0.2 ± | 7.1 ± | 8.0 ± | 4.1 ± |
| | 0.1 | 0.2 | 0.0 | 5.8 |
| 4 | 0.3 ± | 8.9 ± | 10.2 ± | 5.4 ± |
| | 0.1 | 0.1 | 0.1 | 7.7 |
| 5 | 0.0 ± | 10.0 ± | 11.6 ± | 6.3 ± |
| | 0.0 | 0.1 | 0.1 | 8.9 |

TABLE 8
Residual Acetic Acid (g/L)

pH

| Time (d) | 4.5 | 5.00 | 5.50 | 7.00 |
|---|---|---|---|---|
| 0 | 0.3 | 0.3 | 0.3 | 0.3 |
| 1 | 2.0 ± | 1.6 ± | 1.3 ± | 0.5 ± |
| | 0.1 | 0.0 | 0.0 | 0.7 |
| 2 | 1.9 ± | 1.7 ± | 1.3 ± | 0.4 ± |
| | 0.1 | 0.1 | 0.1 | 0.6 |
| 3 | 2.0 ± | 1.5 ± | 1.2 ± | 0.5 ± |
| | 0.3 | 0.1 | 0.0 | 0.6 |
| 4 | 1.9 ± | 1.5 ± | 1.2 ± | 0.5 ± |
| | 0.3 | 0.1 | 0.0 | 0.7 |
| 5 | 2.0 ± | 1.5 ± | 1.2 ± | 0.5 ± |
| | 0.1 | 0.0 | 0.0 | 0.7 |

The results showed that Aurantiochytrium growth was inhibited by internal acetate concentrations of 153 g/L, but not at concentrations of 53 g/L. Therefore, Aurantiochytrium has an acetate toxicity tolerance between about 50 and 150 g/L.

### Example 8

An experiment was conducted to determine the acetate toxicity of Aurantiochytrium sp. HS399. Aurantiochytrium was grown in an acetic acid/pH-auxostat mode in 700 ml bubble column at 27° C. in sterile conditions. The initial culture medium was supplemented with 30 g/L glycerol and 0.5 or 5 g/L acetate. The residual acetic acid was maintained at 1±0.5 g/L and the impact of acetate toxicity on the algae growth was studied at a pH set point of 4.5, 5, 5.5 or 7. Cell dry weights and residual acetate concentration in the culture medium were measured daily, and average internal cell acetate concentration was calculated using the previously described model, and the results are shown in Tables 9, 10, and 11.

TABLE 9
Theoretical Average internal acetate. Based on
an assumed constant intracellular pH and the
residual acetate concentration and medium pH.

| | Average residual | Average internal |
|---|---|---|
| Treatment | acetate (g/L) | acetate (g/L) |
| pH 7.0 | 1.09 | 1.72 |
| pH 5.5 | 1.21 | 51.67 |
| pH 5.0 | 1.34 | 137.06 |
| pH 4.5 | 1.80 | 329.51 |

TABLE 10
Cell Dry Weight (g/L)

pH

| Time (d) | 4.5 | 5.0 | 5.5 | 7.0 |
|---|---|---|---|---|
| 0 | 0.3 ± | 0.3 ± | 0.3 ± | 0.3 ± |
| | 0.0 | 0.0 | 0.0 | 0.0 |
| 1 | 1.8 ± | 2.1 ± | 1.9 ± | 2.7 ± |
| | 0.3 | 0.3 | 0.0 | 0.1 |
| 2 | 4.5 ± | 5.6 ± | 5.5 ± | 7.1 ± |
| | 0.2 | 0.1 | 0.3 | 0.4 |
| 3 | 6.4 ± | 8.1 ± | 8.5 ± | 9.8 ± |
| | 0.1 | 0.0 | 0.4 | 0.4 |
| 4 | 7.8 ± | 9.9 ± | 10.8 ± | 12.0 ± |
| | 0.3 | 0.1 | 0.6 | 0.2 |
| 5 | 8.4 ± | 10.6 ± | 11.4 ± | 12.7 ± |
| | 0.4 | 0.0 | 0.5 | 0.1 |
| 6 | 9.3 ± | 11.4 ± | 12.0 ± | 12.7 ± |
| | 0.3 | 0.0 | 0.4 | 0.0 |

TABLE 11
Residual Acetate (g/L)

pH

| Time (d) | 4.5 | 5.0 | 5.5 | 7.0 |
|---|---|---|---|---|
| 0 | 0.5 ± | 0.5 ± | 0.5 ± | 0.5 ± |
| | 0.0 | 0.0 | 0.0 | 0.0 |
| 1 | 1.8 ± | 1.3 ± | 1.3 ± | 1.1 ± |
| | 0.1 | 0.0 | 0.1 | 0.1 |
| 2 | 1.8 ± | 1.4 ± | 1.3 ± | 1.2 ± |
| | 0.1 | 0.0 | 0.1 | 0.1 |
| 3 | 1.9 ± | 1.4 ± | 1.1 ± | 1.2 ± |
| | 0.1 | 0.1 | 0.3 | 0.1 |
| 4 | 1.8 ± | 1.4 ± | 1.2 ± | 1.2 ± |
| | 0.0 | 0.0 | 0.0 | 0.1 |
| 5 | 1.8 ± | 1.3 ± | 1.2 ± | 1.1 ± |
| | 0.1 | 0.0 | 0.0 | 0.0 |
| 6 | 1.8 ± | 1.3 ± | 1.2 ± | 0.9 ± |
| | 0.1 | 0.0 | 0.0 | 0.0 |

The results showed that Aurantiochytrium growth tolerated 53 g/L, was inhibited by 153 g/l, and did not proliferate at 329 g/L of internal acetate concentration Therefore, Aurantiochytrium has an acetate toxicity tolerance between about 50 and 150 g/L.

### Example 9

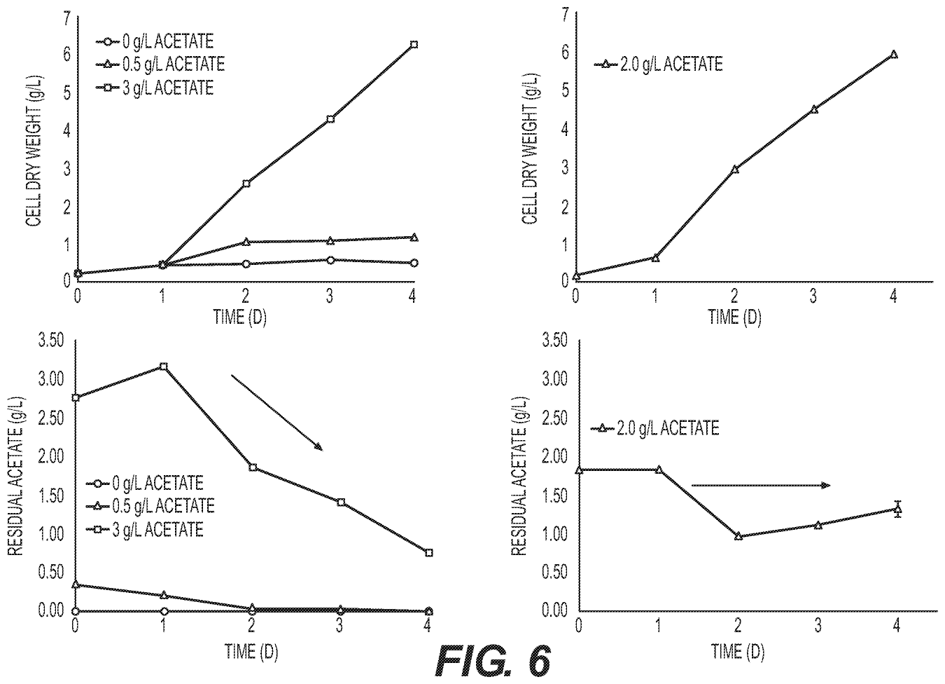

Chlorella sp. HS26 was grown mixotrophically in a 700 ml bubble column, aerated at 1 vvm. Cultures were fed acetic acid in response to pH at a set point of 7.5. An initial concentration of 3-0 g/L sodium acetate was batched to the cultures. The cultures were constantly illuminates at 100 μmol photon/m2 sec. The cultures were grown in a BG-11 were the NO₃ was replaced by NH₄ in an equimolar basis. Two different approaches were used to control residual acetate and acetic acid toxicity and the results are shown in FIG. 6. Initially varying acetic acid batch concentration was attempted, but residual acetic acid concentration in the culture medium (and acetic acid toxicity) decreased over time in response to ammonia assimilation buy the cells. In order to better control acetate toxicity and prevent the depletion of residual acetate in the culture medium (i.e. 0.5 g/L sodium acetate treatment), the feedstock-titrant was modified in order to accommodate acetic acid, NaOH, and NH₄Cl at a 1:0.08:0.14 ratio. The change on the titrant used in the pH auxostat resulted in a better control of residual acetate in the culture medium and ultimately in the better control of the internal cell acetate concentration.

### Example 10

The experiment was conducted with Chlorella sp. HS26 to determine the impact of pH changes on microalgal internal acetate concentration. The treatments were grown in outdoor raceway ponds utilizing a pH auxostat with a set point. Initially the set point was set at pH 7.5 for both treatments, but for one of the treatments pH set point was changed to 6.5 on day 3. Duplicate cultures of each treatment were performed in the experiment. The cultures were supplied a solution of 20% acetic acid, 2% NO3, and 0.79% HCl utilizing the pH auxostat system. The culture media comprised trace nutrients from a BG-11 culturing medium (trace metals formulation available from University of Texas at Austin Culture Collection of Algae (UTEX)) plus+0.5 g/L Sodium Acetate Trihydrate. The cultures were inoculated at a density of 1 g/L, cultured at a temperature of 25° C., and received natural sunlight. Air was sparged into the cultures at a rate of 2.0 m³/hr to maintain a dissolved oxygen concentration of greater than 2 mg O₂/L. Measurements taken every 24 hours included dry weight, residual acetic acid. Cytosolic (internal cell) acetate concentration was calculated using the previously described model. The results are show in Table 12.

TABLE 12

| | Cell Dry | | Residual | | Cytosolic | |
|---|---|---|---|---|---|---|
| | Weight (g/L) | | Acetate (g/L) | | Acetate (g/L) | |
| Time | Constant | Switch pH | Constant | Switch pH | Constant | Switch pH |
| (d) | pH 7.5 | 7.5 → 6.5 | pH 7.5 | 7.5 → 6.5 | pH 7.5 | 7.5 → 6.5 |
| 0 | 1.2 ± | 1.2 ± | 0.1 ± | 0.1 ± | 0.0 ± | 0.0 ± |
| | 0.0 | 0.0 | 0.0 | 0.0 | 0.0 | 0.0 |
| 1 | 1.3 ± | 1.3 ± | 0.4 ± | 0.3 ± | 0.2 ± | 0.2 ± |
| | 0.0 | 0.0 | 0.0 | 0.0 | 0.0 | 0.0 |
| 2 | 2.0 ± | 2.0 ± | 0.4 ± | 0.4 ± | 0.2 ± | 0.2 ± |
| | 0.0 | 0.0 | 0.1 | 0.0 | 0.0 | 0.0 |
| 3 | 2.8 ± | 2.9 ± | 0.6 ± | 0.6 ± | 0.3 ± | 0.3 ± |
| | 0.2 | 0.0 | 0.1 | 0.0 | 0.1 | 0.0 |
| 4 | 4.3 ± | 4.2 ± | 0.4 ± | 0.6 ± | 0.2 ± | 2.8 ± |
| | 0.2 | 0.4 | 0.1 | 0.0 | 0.0 | 0.1 |
| 5 | 5.2 ± | 5.2 ± | 0.3 ± | 0.4 ± | 0.2 ± | 1.9 ± |
| | 0.3 | 0.9 | 0.1 | 0.0 | 0.0 | 0.2 |
| 6 | 5.8 ± | 6.4 ± | 0.4 ± | 0.4 ± | 0.2 ± | 1.9 ± |
| | 0.3 | 0.8 | 0.1 | 0.1 | 0.0 | 0.3 |
| 7 | 5.6 ± | 7.0 ± | 0.4 ± | 0.3 ± | 0.2 ± | 1.7 ± |
| | 1.1 | 0.4 | 0.2 | 0.1 | 0.1 | 0.3 |
| 9 | 5.2 ± | 7.3 ± | 0.2 ± | 0.4 ± | 0.1 ± | 1.9 ± |
| | 2.9 | 0.7 | 0.3 | 0.4 | 0.1 | 1.8 |
| 10 | 5.1 ± | 6.7 ± | 0.2 ± | 0.4 ± | 0.1 ± | 2.1 ± |
| | 2.7 | 1.7 | 0.2 | 0.4 | 0.1 | 1.9 |

As shown in Table 12, the internal cell acetate concentration was substantially constant in the culture that maintained a culture medium pH of 7.5, which is close to the internal cell pH equilibrium of Chlorella. In the treatment where the culture medium pH was lowered to 6.5 to create a pH gradient between the internal cell pH and the pH of the cell culture medium, the internal acetate concentration of less than 500 pm sharply increased to over 2500 ppm in one day and then stabilized at a value above 1500 ppm. These results demonstrate the ability to control the acetate toxicity of a culture by manipulating the culture medium pH and maintaining a substantially constant concentration of acetate in the culture medium.

### Example 11

An experiment was conducted with Chlorella sp. HS26 to evaluate the impact of pH induced acetate toxicity on microalgal growth during a batch culturing process. The two treatments of pH 6.5 and pH 7.5 were set up in outdoor raceway ponds utilizing a pH auxostat with a set point at the designated pH values and cultured for seven days. Duplicate cultures of each treatment were performed in the experiment. The cultures were supplied a solution of 20% acetic acid, 2% NO3, and 0.79% HCl utilizing the pH auxostat system. The culture media comprised trace nutrients from a BG-11 culturing medium (trace metals formulation available from University of Texas at Austin Culture Collection of Algae (UTEX)) plus+0.5 g/L Sodium Acetate Trihydrate. The cultures were inoculated at a density of 1 g/L, cultured at a temperature of 25° C., and received natural sunlight. Air was sparged into the cultures at a rate of 2.0 m³/hr to maintain a dissolved oxygen concentration of greater than 2 mg O₂/L. Measurements taken every 24 hours included dry weight and residual acetic acid in the culture medium. Cytosolic (internal cell) acetate concentration was calculated using the previously described model. The results are show in Table 13.

TABLE 13

| | Cell Dry | | Residual | | Cytosolic | |
|---|---|---|---|---|---|---|
| Time | Weight (g/L) | | Acetate (g/L) | | Acetate (g/L) | |
| (d) | pH 6.5 | pH 7.5 | pH 6.5 | pH 7.5 | pH 6.5 | pH 7.5 |
| 0 | 1.2 ± | 1.2 ± | 0.4 ± | 0.4 ± | 2.1 ± | 0.2 ± |
| | 0.0 | 0.0 | 0.0 | 0.0 | 0.2 | 0.0 |
| 1 | 1.3 ± | 1.2 ± | 0.5 ± | 0.5 ± | 2.6 ± | 0.2 ± |
| | 0.0 | 0.0 | 0.0 | 0.0 | 0.1 | 0.0 |
| 2 | 1.5 ± | 1.4 ± | | | | |
| | 0.2 | 0.0 | | | | |
| 3 | 1.7 ± | 1.8 ± | | | | |
| | 0.6 | 0.0 | | | | |
| 4 | 2.6 ± | 2.7 ± | 0.4 ± | 0.3 ± | 2.1 ± | 0.2 ± |
| | 1.3 | 0.1 | 0.1 | 0.1 | 0.6 | 0.0 |
| 5 | 4,1 ± | 3.9 ± | | | | |
| | 2.0 | 0.1 | | | | |
| 6 | 4.7 ± | 5.1 ± | 0.2 ± | 0.2 ± | 1.0 ± | 0.1 ± |
| | 1.4 | 0.2 | 0.2 | 0.1 | 1.0 | 0.0 |

As shown in Table 13, the cultures at pH 6.5 lasted for 5 days. The average three-day productivity for the pH 6.5 cultures (1.14 g/L/day) was higher than for the pH 7.5 cultures (0.76 g/L/day), and demonstrate the benefits of applying the concept of acetate toxicity to induce the uptake of acetate by the cell. The induced uptake of the acetate (i.e., organic carbon) is shown in the calculated internal cell acetate concentration and the increased metabolic activity that resulted in an increase in productivity.

### Example 12

The experiment of Example 11 was repeated with Chlorella sp. HS26 to evaluate the impact of pH induced acetate toxicity on microalgal lipid and protein accumulation. The two treatments of pH 6.5 and pH 7.5 were set up in outdoor raceway ponds utilizing a pH auxostat with a set point at the designated pH values. The microalgae were cultured for 8 days. Duplicate cultures of each treatment were performed in the experiment. The cultures were supplied a solution of 20% acetic acid, 2% NO₃, and 0.79% HCl utilizing the pH auxostat system. The culture media comprised trace nutrients from a BG-11 culturing medium (trace metals formulation available from University of Texas at Austin Culture Collection of Algae (UTEX)) plus+0.5 g/L Sodium Acetate Trihydrate. The cultures were inoculated at a density of 1 g/L, cultured at a temperature of 25° C., and received natural sunlight. Air was sparged into the cultures at a rate of 2.0 m³ hr to maintain a dissolved oxygen concentration of greater than 2 mg O₂/L. The results of the total lipids, protein, carbohydrates, and ash at the beginning and end of the experiment are show in Table 14.

TABLE 14

| | | Total | Total | Total | |
|---|---|---|---|---|---|
| | Treatment | Protein | Lipids | Carbohydrates | Ash |
| Time (d) | pH | (% DW) | (% DW) | (% DW) | (% DW) |
| 0 | 6.5 | 35.07 ± | 9.82 ± | 28.51 ± | 26 6 ± |
| | | 0.0 | 0.011 | 0.0 | 1.48 |
| | 7.5 | 35.07 ± | 9.82 ± | 28.51 ± | 26 6 ± |
| | | 0.0 | 0.011 | 0.0 | 1.48 |
| 8 | 6.5 | 35.28 ± | 29.34 ± | 32.13 ± | 3.26 ± |
| | | 0.39 | 1.69 | 2.0 | 0.09 |
| | 7.5 | 39.69 ± | 23.66 ± | 33.62 ± | 3.05 ± |
| | | 0.13 | 0.06 | 0.19 | 0.12 |

A shown in Table 14, the pH 6.5 cultures accumulated more lipids and less protein than the pH 7.5 cultures.

### Example 13

An experiment was conducted to determine the effect of different nitrogen sources on the internal cell acetate concentration for cultures of Aurantiochytrium sp. HS399 cultures. Aurantiochytrium was grown in a 100 L open pond using acetic acid as only carbon source. Acetic acid was fed in response to pH by a pH auxostat system, while controlling the set point at 5.5. One treatment used monosodium glutamate (MSG) and another treatment used ammonia (NH₃) as a nitrogen source. The base medium contained (g/L): sodium acetate (1 g/L), monosodium glutamate monohydrate (5.5) NaCl (12.5), MgSO4. 7H2O (2.5), KCl (0.5), CaCl2 (0.1), KH2PO4 (0.5) vitamins (1 ml/L) and trace metal (5 ml/L). Trace and vitamins stocks were prepared according to Ashford, et al. 2000. Lipids 35, 1377-1386. The ponds were inoculated at 1 v/v using exponentially growing cultures. Aeration was maintained at 0.5 vvm using a porous hose. Equimolar nitrogen concentrations were added top each pond reactor. In order to avoid residual acetate running low, the MSG treatment was batched with 1 g/L sodium acetate, while the NH₃ treatment was blended with 3.5 g/L of acetic acid. Culture dry weights (filtration) and residual acetate (HPLC) in the culture medium were analyzed daily. Cytosolic (internal cell) acetate concentration was calculated using the previously described model. The results are shown in Table 15.

TABLE 15

| | Cell Dry | | Residual | | Cytosolic | |
|---|---|---|---|---|---|---|
| Time | Weight (g/L) | | Acetate (g/L) | | Acetate (g/L) | |
| (d) | MSG | NH3 | MSG | NH3 | MSG | NH3 |
| 0 | 0.2 ± | 0.2 ± | 0.6 ± | 3.3 ± | 24.1 ± | 140.9 ± |
| | 0.0 | 0.0 | 0.0 | 0.1 | 0.3 | 6.0 |
| 1 | 3.5 ± | 1.9 ± | 2.5 ± | 2.2 ± | 104.6 ± | 93.9 ± |
| | 0.0 | 0.6 | 0.1 | 0.3 | 3.0 | 12.1 |
| 2 | 6.8 ± | 7.4 ± | 2.2 ± | 1.2 ± | 93.9 ± | 51.2 ± |
| | 0.2 | 0.5 | 0.1 | 0.0 | 6.0 | 0.0 |
| 3 | 8.1 ± | 7.8 ± | 1.9 ± | 1.1 ± | 81.1 ± | 47.8 ± |
| | 1.0 | 1.4 | 0.0 | 0.3 | 0.0 | 10.9 |
| 4 | 9.7 ± | 9.9 ± | 1.6 ± | 1.2 ± | 68.3 ± | 49.3 ± |
| | 1.9 | 1.1 | 0.3 | 0.3 | 12.1 | 14.8 |
| 5 | 10.2 ± | 10.3 ± | 1.4 ± | 1.2 ± | 59.8 ± | 52.7 ± |
| | 2.8 | 1.5 | 0.6 | 0.4 | 24.2 | 16.0 |

As shown in Table 15, under the acetic acid/pH auxostat fed-batch configuration the MSG initially increased the internal cell acetate concentration while the ammonia treatment showed a consistent decrease in the internal acetate concentration. While the growth data was similar for both treatments, the data in Table 15 shows the choice of nitrogen source results in a difference in internal cell acetate concentration and may be another variable for manipulating the acetate toxicity conditions for a commercial process.

### Example 14

An experiment was conducted to determine the effectiveness of utilizing an additional acid in combination with acetic acid to control the culture medium pH and residual acetate concentration of the culture medium, which correspond to the acetate toxicity conditions. Chlorella sp. HS26 was grown in an acetic acid/pH-auxostat mode in 700 ml bubble column at 25 C, pH 7.5 and sterile conditions. The BG-11 medium was supplemented with 0.5 g/L sodium acetate and aerated at 1 vvm. Two treatments were tested in quadruplicates, one was supplied with acetic acid only and the second one was supplied with a blend of acetic acid and hydrochloric acid (HCl) in a ratio of 20:0.8 w/w. Samples were taken daily to measure dry weight and residual acetate concentration of the culture medium. The results are show in Table 16.

TABLE 16

| | Cell Dry | | Residual | | Cytosolic | |
|---|---|---|---|---|---|---|
| | Weight (g/L) | | Acetate (g/L) | | Acetate (g/L) | |
| | | Acetic | | Acetic | | Acetic |
| Time | Acetic | Acid/HCl | Acetic | Acid/HCl | Acetic | Acid/HCl |
| (d) | Acid | 20:0.8 | Acid | 20:0.8 | Acid | 20:0.8 |
| 0 | 0.3 | 0.3 | 0.2 | 0.2 | 0.1 | 0.1 |
| 1 | 0.8 ± | 0.8 ± | 0.5 ± | 0.4 ± | 0.2 ± | 0.2 ± |
| | 0.0 | 0.0 | 0.0 | 0.0 | 0.0 | 0.0 |
| 2 | 2.7 ± | 2.6 ± | 0.9 ± | 0.6 ± | 0.5 ± | 0.3 ± |
| | 0.1 | 0.1 | 0.0 | 0.0 | 0.0 | 0.0 |
| 3 | 3.7 ± | 3.6 ± | 1.1 ± | 0.6 ± | 0.6 ± | 0.3 ± |
| | 0.1 | 0.1 | 0.0 | 0.0 | 0.0 | 0.0 |
| 4 | 4.7 ± | 4.6 ± | 1.4 ± | 0.6 ± | 0.7 ± | 0.3 ± |
| | 0.1 | 0.1 | 0.0 | 0.0 | 0.0 | 0.0 |
| 5 | 5.3 ± | 5.1 ± | 1.6 ± | 0.7 ± | 0.8 ± | 0.3 ± |
| | 0.1 | 0.1 | 0.1 | 0.0 | 0.0 | 0.0 |
| 6 | 5.3 ± | 5.2 ± | 1.6 ± | 0.7 ± | 0.8 ± | 0.3 ± |
| | 0.3 | 0.2 | 0.1 | 0.0 | 0.1 | 0.0 |

As shown in Table 16, the addition of HCl was effective to maintain the culture conditions to produce similar growth as the acetic acid only treatment, but also maintained the residual acetate concentration of the culture medium at a lower level that the acetic acid only treatment. These results demonstrate that the addition of HCl with an acetic acid pH auxostat system allows for better control over the residual acetate concentration of the culture medium, and thus facilitates the ability to apply the concept of acetate toxicity to a microalgae culture for a targeted benefit, such as increased secondary metabolites (e.g., lipids).

### Example 15

An experiment was performed to determine the effectiveness of applying acetate toxicity to microalgae cultures grown in the presence of a non-acetate organic carbon source, in this case glycerol. Aurantiochytrium sp. HS399 was grown in a 100 L open pond. The base medium contained: sodium acetate (1 g/L), glycerol (30 g/L) monosodium glutamate monohydrate (2.5 g/L) NaCl (12.5 g/L), MgSO4.7H2O (2.5 g/L), KCl (0.5 g/L), CaCl₂) (0.1 g/L), KH2PO4 (0.5 g/L), vitamins (1 ml/L), and trace metal (5 ml/L). Trace and vitamins stocks were prepared according to Ashford, et al. 2000. Lipids 35, 1377-1386. The ponds were inoculated at 1 v/v using exponentially growing cultures. Aeration was maintained at 0.5 vvm using a porous hose. In one treatment the pH was controlled at a set point of pH 7 with hydrochloric acid, and no acetic acid was supplied. In another treatment acetic acid was supplied by a pH auxostat system at a set point of 5.5 The impact on the culture of acetate toxicity conditions created by the pH and residual acetate concentration in the medium in microalgae growth and contamination was analyzed by measuring dry weights (filtration), residual acetate concentration in the culture medium (HPLC), and total aerobic bacteria counts (petrifilms) daily. Cytosolic (internal cell) acetate concentration was calculated using the previously described model. The results are shown in Tables 17 and 18.

TABLE 17

| | Cell Dry | | Residual | | Cytosolic | |
|---|---|---|---|---|---|---|
| | Weight (g/L) | | Acetate (g/L) | | Acetate (g/L) | |
| | pH 5.5, | | pH 5.5, | | pH 5.5, | |
| Time | Acetic | pH 7.0, | Acetic | pH 7.0, | Acetic | pH 7.0, |
| (d) | Acid | HCl | Acid | HCl | Acid | HCl |
| 0 | 0.0 ± | 0.1 ± | 0.5 ± | 0.0 ± | 23.1 ± | 0.0 ± |
| | 0.0 | 0.0 | 0.0 | 0.0 | 1.2 | 0.0 |
| 1 | 1.1 ± | 1.6 ± | 1.0 ± | 0.0 ± | 44.0 ± | 0.0 ± |
| | 0.1 | 0.0 | 0.1 | 0.0 | 4.2 | 0.0 |
| 2 | 3.5 ± | 4.3 ± | 1.4 ± | 0.0 ± | 57.6 ± | 0.0 ± |
| | 0.4 | 0.5 | 0.1 | 0.0 | 3.0 | 0.0 |
| 3 | 6.2 ± | 5.5 ± | 1.5 ± | 0.0 ± | 64.0 ± | 0.1 ± |
| | 0.6 | 0.9 | 0.0 | 0.0 | 0.0 | 0.1 |
| 4 | 7.7 ± | 6.5 ± | 0.9 ± | 0.1 ± | 39.7 ± | 0.1 ± |
| | 0.1 | 1.4 | 0.7 | 0.0 | 28.4 | 0.0 |
| 5 | 9.7 ± | 8.1 ± | 1.1 ± | 0.0 ± | 46.3 ± | 0.0 ± |
| | 0.4 | 1.2 | 0.7 | 0.0 | 31.1 | 0.0 |

TABLE 18

Contamination (CFU/mL)

| | pH 5.5, | |
|---|---|---|
| Time (d) | Acetic Acid | pH 7.0, HCl |
| 0 | 5.5E+04 | 1.9E+05 |
| 1 | 4.8E+05 | 2.9E+10 |
| 2 | 5.7E+07 | 1.3E+16 |
| 3 | 4.0E+10 | 2.1E+18 |
| 4 | 4.6E+12 | 1.4E+17 |
| 5 | 8.0E+14 | 3.4E+21 |

As shown in Table 17, the culture in acetate toxicity conditions had comparable growth to the other culture. As shown in Table 18, the aerobic bacteria counts decreased at least by 3 log throughout the batch for the culture in acetate toxicity conditions as compared to the other treatment.

### Example 16

A experiment was performed to demonstrate that a method of controlling acetate toxicity is successful with non-axenic culture of the heterotrophic microalgae Aurantiochytrium sp. (HS399) in open outdoor bioreactor. An axenic inoculum culture of Aurantiochytrium was produced in bag bioreactors and transferred to a non-sterile open raceway pond bioreactor with a volume of about 18,000 L disposed outdoors. The non-axenic culture was cultured from an inoculation density of about 0.2 g/L until the culture density reached 10-20 g/L. The open bioreactor culture received acetic acid through a pH auxostat system where the pH set point was 5.5. Results of the culturing in the open bioreactor stage are shown in Tables 19, 20, 21, and 22.

TABLE 19

| | Cell Dry | |
|---|---|---|
| Time (d) | Weight (g/L) | Lipids (g/L) |
| 0.0 | 0.3 | 0.2 |
| 0.2 | 0.4 | |
| 0.7 | 2.8 | 2.4 |
| 1.2 | 4.8 | |
| 1.7 | 9.4 | |
| 2.0 | 10.3 | 8.0 |
| 2.2 | 11.1 | |

TABLE 20

| | Cumulative Acetic | Residual Acetic |
|---|---|---|
| Time (d) | Acid Input (g/L) | Acid (g/L) |
| 0.0 | 0.5 | 0.7 |
| 0.9 | 13.4 | 1.6 |
| 1.3 | 19.2 | |
| 1.9 | 34.6 | |
| 1.9 | 35.9 | 1.7 |
| 2.1 | 38.8 | |

TABLE 21

| | Lean Biomass | Total Fatty | DHA |
|---|---|---|---|
| Time (d) | (g/L) | Acids (g/L) | (g/L) |
| 0.0 | 0.1 | 0.1 | 0.0 |
| 0.9 | 2.0 | 1.4 | 0.4 |
| 1.9 | 2.6 | 5.6 | 1.7 |
| 2.0 | 2.3 | 6.2 | 1.9 |

TABLE 22

| | Aerobic bacteria |
|---|---|
| Time (d) | count (CFU/mL) |
| 0.0 | 1.1E+04 |
| 0.9 | 2.1E+04 |
| 1.9 | 2.2E+04 |

As show in Tables 19-22, the acetate toxicity conditions resulted in successful microalgae growth, lipid accumulation, and management of the bacteria population in open outdoor conditions.

### Example 17

A experiment will be performed in which a mixotrophic or heterotrophic microalgae will be cultured in a culture wherein the culture receives as a feedstock or the culture medium comprises a refined or unrefined by-product stream from industrial, municipal, or agricultural sources may comprise ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, industrial waste solutions, yeast extract, pigments, nitrates, phosphates, phosphites, and combinations thereof. The microalgae culture will receive a supply of at least one of acetate and acetic acid. The acetate concentration and culture pH levels will be controlled to maintain an acetate toxicity level that is permissive for microalgae growth conditions while suppressing the metabolic activity of bacteria in the culture. The culture dry weight, pH, residual acetate concentration, and acetate consumption will be monitored in the culture. The microalgae culture growth rate, length of the microalgae culture life, and resulting biomass will be compared to cultures that do not receive acetate/acetic acid or do not control the acetate toxicity within the desired band for the particular microalgae.

### Exemplary Aspects of the Invention

In one non-limiting embodiment of the invention, a method of managing acetate toxicity in a microalgae culture is provided, which method may comprise: providing a culture comprising microalgae: supplying the culture with at least one of acetate and acetic acid; optionally measuring a pH of the culture medium and a residual acetate concentration in the culture medium; and controlling the pH of the culture medium and the residual acetate concentration in the culture medium to maintain an internal microalgae cell acetate concentration within a calculated range to provide at least one measurable/detectable benefit to the microalgae culture. The measurable benefit can include a) detectably inhibiting the growth of bacteria in the culture (or imparting the capacity to detectably inhibit the growth of bacteria in the culture in the event of bacterial contamination of the culture where the culture is axenic), (b) detectably increase the production of one or more secondary metabolites in the culture, (c) detectably increasing the growth rate of the microalgae cell population, (d) detectably decrease the production of one or more macronutnents, (e) detectably increase the average cellular respiration rate of the microalgae, (f) detectably increasing the metabolic rate of the microalgae, (g) detectably increasing acetate uptake of the microalgae, (h) detectably increasing the productive life of the culture, or a combination of any or all of (a)-(h).

In some aspects, detectably increasing the growth rate of the microalgae cell population means increasing the growth (as measured by increase in biomass) of the culture at least 2 fold (100%), at least 3 fold, at least 4 fold, at least 5 fold, at least 6 fold, at least 7 fold, at least 7.25 fold, at least 7.5 fold, at least 8 fold, at least 9 fold, at least 10 fold, or even more such as at least 12 fold, at least 15 fold, at least 20 fold (e.g., at least 21, at least 22, at least 23, at least 24, at least 25, at least 26, or at least 27 fold), or more (such as at least 30 fold, at least 35 fold, at least 40 fold, at least 45 fold, at least 50 fold, at least 55 fold, or even at least 60 fold) as compared to a control culture, as measured over a period of 1, 2, 3, 4, 5, 6, 7, 8, 9, 10, 11, 12, 13, 14, 15, or more (up to 20) days, as, for example, exemplified in the Examples. Typically the culture is maintained for a period of 4-20, such as 5-15, such as 6-14 or 7-14 days, and the enhancement of growth rate is measured at the end of such period.

In another aspect, the benefit is an increase in the production of one or more lipid secondary metabolites and the increase is at least 33%, at least 40%, at least 50%, at least 65%, at least 75%, at least 100% (2× or two-fold), at least 125%, at least 150%, at least 175%, at least 200%, at least 225%, at least 233%, or more (e.g., at least 240%). The increase can be measured by total lipid production or production of select lipids, such as omega-3 fatty acids (e.g., DHA). In other aspects practice of the method also or alternatively can result in an increase in fatty acid content in the culture of at least two-fold (100%), such as at least 150%, at least 200%, at least 250%, at least 300%, at least 400%, at least 500%, or even more such as at least 10-fold, at least 15-fold, at least 20-fold, at least 25-fold, at least 30-fold, at least 35-fold, at least 40-fold, at least 45-fold, at least 50-fold, at least 55-fold, or even at least 60-fold.

In still another aspect, the culture comprises bacteria and the practice of the method inhibits the growth of bacteria such that the increase in the number of bacteria cells throughout the productive life of the culture is restricted to a 100% (2-fold) increase or less, such as a 75% increase or less, a 65% increase or less, a 50% increase or less, or a 25% increase or less. In still another aspect, the method increases the useful or productive life of the culture by a period of time that can be 1, 2, or more days, or a percentage of time corresponding to such an increase of days. The productive or useful culture life is determined by the period that the microalgae is continuing to increase in one or more of the beneficial properties related to microalgae product production and/or growth described herein, such as biomass, lipid production, and the like. Once the maximum benefit is achieved and/or the amount of such a benefit begins to decline the culture is typically harvested (in whole or in part) or otherwise terminated.

It should be noted that with respect to many of the measurements described herein with respect to characterizing microalgae of the culture that such values can mean an average value of microalgae cells in the culture (e.g., for acetate update), or can mean a minimum value that can be applied to the entire culture (at least within limits of current detection methods), or can mean that a substantial proportion of the culture has such characteristic (i.e., at least 25%, such as at least 33%, at least 35%, or at least 40%) of the cells in the culture have the characteristic; a majority of cells in the culture have the characteristic: or a predominate portion of the culture (at least 66.333%, such as at least 75%, at least 80%, at least 85%, at least 90%, at least 95%, or more) of the cells in the culture have the method (in some cases 99%+ or even all detectable cells have the characteristic). In alternative aspects, the characteristic is present in at least a detectable number of cells in the culture.

In some embodiments, the step of controlling the pH of the culture medium may further comprise the addition of acetic acid. In some embodiments, the step of controlling the pH of the culture medium may further comprise the addition of a second acid different from acetic acid. In some embodiments, the second acid may comprise at least one selected from the group consisting of hydrochloric acid, phosphoric acid, and sulfuric acid.

In some embodiments, the microalgae may use the supply comprising at least one of acetate and acetic acid as an organic carbon source. In some embodiment, the microalgae culture may be further supplied with at least one organic carbon source selected from the group consisting of ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

In some embodiments, the microalgae may be Chlorella. In some embodiments, the internal Chlorella cell acetate concentration may be maintained in the range of 6-11 g/L. In some embodiments, the microalgae may be Aurantiochytrium. In some embodiments, the internal Aurantiochytrium cell acetate concentration may be maintained in the range of 50-150 g/L.

In some embodiments, the culture may further comprise bacteria and the at least one benefit to the microalgae culture may comprise inhibiting the growth of bacteria. In some embodiments, the at least one benefit to the microalgae culture may comprise an increase in growth rate. In some embodiments, the at least one benefit to the microalgae culture may comprise an increase in secondary metabolite accumulation. In some embodiments, the secondary metabolite may be lipids. In some embodiments, the lipids may be accumulated in nitrogen sufficient conditions. In some embodiments, the lipids may be accumulated in nitrogen deficient conditions.

In some embodiments, the at least one benefit to the microalgae culture may comprise an increase in the uptake of acetate. In some embodiments, the at least one benefit to the microalgae culture may comprise an increase in the metabolic rate of the microalgae. In some embodiments, the at least one benefit to the microalgae culture may comprise an increase in the respiration rate. In some embodiments, the at least one benefit may comprise a reduction in the culture time for production of a secondary metabolite. In some embodiments, the at least one benefit to the microalgae culture may comprise an increase in the maximum culture density in a closed bioreactor culture. In some embodiments, the at least one benefit to the microalgae culture may comprise a decrease in the accumulation of protein. In some embodiments, the at least one benefit to the microalgae culture may comprise a decrease in the accumulation of a macronutrient, such as one or more carbohydrates, proteins, or a combination thereof (although in other aspects one or both of these macronutrients can be increased by the practice of the invention).

In some embodiments, the at least one benefit to the microalgae culture may comprise an increased culture life in a culture medium comprising refined or unrefined by-product streams from industrial, municipal, or agricultural sources. In some embodiments, the at least one benefit to the microalgae culture may comprise an increase in the culture life when in the presence of contaminating organisms.

In some embodiments, the culture may comprise monosodium glutamate as a nitrogen source. In some embodiments, the culture may comprise at least one of ammonia and ammonium as a nitrogen source.

### INTERPRETATION, REFERENCES, AND MISCELLANEOUS

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about.” where appropriate). All provided ranges of values are intended to include the end points of the ranges, as well as values between the end points.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

### REFERENCES

Liang, et al. Utilization of acetic acid-rich pyrolytic bio-oil by microalga Chlamydomonas reinhardtii: reducing bio-oil toxicity and enhancing algal toxicity tolerance. Bioresource Technology, 2013 April, 133:500-6. doi: 10.1016/j.biortech.2013.01.134. Epub 2013 Feb. 9
Roberts, et al. Toxicity of haloacetic acids to freshwater algae. Ecotoxicology and Environmental Safety, Volume 73, Issue 1, January 2010, Pages 56-61.
http://www.amnh.org/leam-teach/young-naturalist-awards/winning-essays2/2014-winning-essays/algae-a-truly-green-fuel-to-power-the-world
Greenway, et al. Effects of low water potentials on respiration and on glucose and acetate uptake, by Chlorella pyrenoidosa. Planta, 16, June 1967, Volume 75, Issue 3, pp. 253-374
Syrett, et al. The Assimilation of Acetate by Chlorella vulgaris. Journal of Experimental Botany, 1964, Volume 15, Issue 1, Pages 35-47
Fujita, Kazuko. The metabolism of acetate in Chlorella Cells. The Journal of Biochemistry, (1959), Volume 46, Issue 3, Page 253-268.
Matsuka, et al. Acetate metabolism in the process of “acetate-bleaching” of Chlorella protothecoides. Plant and Cell Physiology, 1969, Volume 10, Issue 3. Pages 527-538.
WO2014016612A1
WO2010080377A1
US20140038247
US20120088279
U.S. Pat. No. 8,778,643

1. A method comprising:
a. Providing a culture comprising a population of microalgae cells, the microalgae cells having an acetate toxicity threshold level;
b. Supplying the culture with at least one of acetate and acetic acid; and
c. Controlling the pH of the culture medium and residual acetate concentration in the culture medium to maintain an average internal microalgae cell acetate concentration below the acetate toxicity threshold level so as to (a) detectably inhibit the growth of bacteria in the culture, (b) detectably increase the production of one or more secondary metabolites in the culture, (c) detectably increasing the growth rate of the microalgae cell population, (d) detectably decrease the production of one or more macronutrients, (e) detectably increase the average cellular respiration rate of the microalgae, (f) detectably increasing the metabolic rate of the microalgae, (g) detectably increasing acetate uptake of the microalgae, (h) detectably increasing the productive life of the culture, or a combination of any or all of (a)-(h).

2. The method of claim 1, wherein the step (c) further comprises adding acetic acid to the culture.

3. The method of claim 1, wherein the step (c) further comprises addition of a second acid that is different from acetic acid.

4. The method of claim 3, wherein the second acid comprises at least one acid selected from the group consisting of hydrochloric acid, phosphoric acid, and sulfuric acid.

5. The method of claim 1, wherein the microalgae uses the supply comprising at least one of acetate and acetic acid as an organic carbon source.

6. The method of claim 1, further supplying the microalgae culture with at least one organic carbon source selected from the group consisting of ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, agricultural by-products, industrial process by-products, municipal waste streams, yeast extract, and xylose.

7. The method of claim 1, wherein the microalgae comprises Chlorella.

8. The method of claim 1, wherein the average internal microalgae cell acetate concentration is maintained at up to 11 g/L.

9. The method of claim 1, wherein the microalgae comprises Aurantiochytrium.

10. The method of claim 1, wherein the average internal microalgae cell acetate concentration of at least one species of microalgae in the culture is maintained at up to 150 g/L.

11. The method of claim 1, wherein the culture further comprises bacteria and the at least one benefit to the microalgae culture comprises inhibiting the growth of bacteria.

12. The method of claim 1, wherein the method comprises increasing the production of one or more secondary metabolites in the culture, where at least some of the secondary metabolites comprise lipids.

13. The method of claim 1, wherein the method comprises maintaining the culture in nitrogen sufficient conditions.

14. The method of claim 1, wherein the method comprises maintaining the culture in nitrogen deficient conditions.

15. The method of claim 1, wherein the method results in the decrease of a macronutrient in the microalgae that is selected from the group consisting of protein, carbohydrate, and a combination thereof.

16. The method of claim 1, wherein the culture comprises one or more by-product streams from industrial, municipal, or agricultural sources.

17. The method of claim 1, wherein the culture comprises one or more contaminating organisms and practice of the method results in increase in the productive culture life of the microalgae in the presence of contaminating organisms.

18. The method of claim 1, further comprising adding monosodium glutamate to the culture as a nitrogen source.

19. The method of claim 1, further comprising at least one of ammonia and ammonium as a nitrogen source to the culture.

20. A method of culturing microalgae in acetate toxicity conditions comprising:
a. Providing a culture comprising a population of microalgae cells;
b. Determining an acetate toxicity threshold level for the microalgae cells;
c. Supplying the culture with at least one of acetate and acetic acid; and
d. Controlling the pH of the culture medium and residual acetate concentration in the culture medium to maintain an average internal microalgae cell acetate concentration below the acetate toxicity threshold level so as to (a) detectably inhibit the growth of bacteria in the culture, (b) detectably increase the production of one or more secondary metabolites in the culture, (c) detectably increasing the growth rate of the microalgae cell population, (d) detectably decrease the production of one or more macronutrients, (e) detectably increase the average cellular respiration rate of the microalgae, (f) detectably increasing the metabolic rate of the microalgae, (g) detectably increasing acetate uptake of the microalgae, (h) detectably increasing the productive life of the culture, or a combination of any or all of (a)-(h).

21. The method of claim 20, wherein the microalgae comprises Chlorella.

22. The method of claim 21, wherein the acetate toxicity threshold level of Chlorella at a pH of 7 is in the range of 6-11 g/L and the average internal microalgae cell acetate concentration is maintained below the acetate toxicity threshold level of Chlorella.

23. The method of claim 20, wherein the microalgae comprises Aurantiochytrium.

24. The method of claim 23, wherein the acetate toxicity threshold level of Aurantiochytrium at a pH of about 7 is in the range of 50-150 g/L and the average internal microalgae cell acetate concentration culture is maintained below the acetate toxicity threshold level of Aurantiochytrium.
